(19) United States
(12) Patent Application Publication
(10) Pub. No.: US 2020/0330646 A1
(43) Pub. Date: Oct. 22, 2020

(54) SCAFFOLDS HAVING MATERIAL PROPERTIES OPTIMIZED FOR CARDIAC APPLICATIONS AND USES THEREOF

(71) Applicant: Arizona Board of Regents on Behalf of the University of Arizona, Tucson, AZ (US)

(72) Inventors: Steven Goldman, Tucson, AZ (US); Jordan Lancaster, Tucson, AZ (US); Jennifer Koevary, Tucson, AZ (US)

(21) Appl. No.: 16/764,536

(22) PCT Filed: Nov. 16, 2018
(86) PCT No.: PCT/US2018/061568

§ 371 Date: May 15, 2020

Related U.S. Application Data
(60) Provisional application No. 62/588,043, filed on Nov. 17, 2017.

(51) Int. Cl. A61L 27/38; A61L 27/18; A61L 27/54; A61L 27/58; A61K 35/34; A61K 47/34; A61L 27/56

(52) U.S. Cl. CPC A61L 27/3873; A61L 27/3834; A61L 27/18; A61L 27/54; A61L 2430/20; A61K 35/34; A61K 47/34; A61L 27/56; A61L 27/58

(57) ABSTRACT

Provided herein are scaffolds (e.g., synthetic meshes) having optimized material properties (e.g., initial stiffness, tensile strength) and related uses thereof (e.g., use in cardiac medical procedures).

### CROSS REFERENCE TO RELATED APPLICATION

This application claims priority to and the benefit of U.S. Provisional Application No. 62/588,043, filed Nov. 17, 2017, which is hereby incorporated by reference in its entirety.

### FIELD

Provided herein are scaffolds (e.g., synthetic meshes) having optimized material properties (e.g., initial stiffness, tensile strength) and related uses thereof (e.g., use in cardiac medical procedures).

### BACKGROUND

Heart conditions such as arrhythmia, cardiomyopathies and congenital defects are widespread. For example, chronic heart failure (CHF) is one of the leading causes of death in the United States, affecting more than 15 million people Signs and symptoms of heart failure commonly include shortness of breath, excessive tiredness, and leg swelling. The shortness of breath is usually worse with exercise, while lying down, and may wake the person at night. A limited ability to exercise is also a common feature. Chest pain, including angina, does not typically occur due to heart failure.

Common causes of heart failure include coronary artery disease including a previous myocardial infarction (heart attack), high blood pressure, atrial fibrillation, valvular heart disease, excess alcohol use, infection, and cardiomyopathy of an unknown cause. These cause heart failure by changing either the structure or the functioning of the heart. The two types of heart failure—heart failure with reduced ejection fraction (HFrEF), and heart failure with preserved ejection fraction (HFpEF)—are based on whether the ability of the left ventricle to contract is affected, or the heart's ability to relax. The severity of disease is graded by the severity of symptoms with exercise. Heart failure is not the same as myocardial infarction (in which part of the heart muscle dies) or cardiac arrest (in which blood flow stops altogether). Heart failure is diagnosed based on the history of the symptoms and a physical examination, with confirmation by echocardiography. Blood tests, electrocardiography, and chest radiography may be useful to determine the underlying cause.

Treatment depends on the severity and cause of the disease. In people with chronic stable mild heart failure, treatment commonly consists of lifestyle modifications such as stopping smoking, physical exercise, and dietary changes, as well as medications. In those with heart failure due to left ventricular dysfunction, angiotensin converting enzyme inhibitors, angiotensin receptor blockers, or valsartan/sacubitril along with beta blockers are recommended. For those with severe disease, aldosterone antagonists, or hydralazine with a nitrate may be used. Diuretics are useful for preventing fluid retention. Sometimes, depending on the cause, an implanted device such as a pacemaker or an implantable cardiac defibrillator (ICD) may be recommended. In some moderate or severe cases, cardiac resynchronization therapy (CRT) or cardiac contractility modulation may be of benefit. A ventricular assist device or occasionally a heart transplant may be recommended in those with severe disease that persists despite all other measures.

There is a need for additional treatments for CHF, especially severe CHF.

The present invention addresses this need.

### SUMMARY

Experiments conducted during the course of developing embodiments for the present invention conducted in vivo and in vitro experiments on commercially available biodegradable scaffolds having varying levels of degradation for purposes of identifying the optimized material properties of such scaffolds for cardiac applications. Such experiments used in vivo pressure volume loops to identify materials that did not restrict cardiac filling and used mechanical testing of materials to identify the initial stiffness and initial tensile strength values for materials that did not restrict cardiac filling.

Accordingly, in certain embodiments, the present invention provides scaffolds configured for implanting in a human or animal body, wherein the scaffold has an initial stiffness value and initial mechanical tensile strength value optimized for cardiac application.

Such scaffolds are not limited to particular initial stiffness values and/or initial mechanical strength values. In some embodiments, the initial stiffness values and/or initial mechanical strength values enable the resulting scaffold to engage with heart tissue and not adversely affect native cardiac function. In some embodiments, the initial stiffness values and/or initial mechanical strength values prevent a prolonged degradation period. In some embodiments, the initial stiffness values and/or initial mechanical strength values prevent restriction of ventricular filling.

In some embodiments, the scaffold does not shift the pressure-volume loop toward the pressure axis from normal.

In some embodiments, the scaffold has an initial stiffness of at or lower than 40 N/mm (e.g., lower than 30.0, 10.0, 6.0, 4.0, 3.0, or 2.0).

In some embodiments, the scaffold has an initial mechanical tensile at or lower than approximately 85N.

In some embodiments, the scaffold comprises synthetic material. In some embodiments, the scaffold comprises biological material. In some embodiments, the scaffold is a hybrid of synthetic and biological materials.

In some embodiments, the scaffold is composed of one or more of collagen, fibronectin, polyglycolides, polylactides, polypropylene, polyester, silicone, expanded polytetrafluorothylene, Dexon, Vicryl, polycaprolactone, polycarbonate, polydioxanone, catgut, silk, nylon, and trimethylene carbonate. In some embodiments, the scaffold is composed of a polylactide material. In some embodiments, the scaffold is composed of a polyglactin 910 material. In some embodiments, the scaffold is derived from human, bovine or porcine tissue.

In some embodiments, the scaffold is bioabsorbable. In some embodiments, the scaffold is non-bioabsorbable.

In some embodiments, the scaffold further comprises a therapeutic agent such as a drug or biologic. In some embodiments, the therapeutic agent is known to be useful in treating, ameliorating and/or preventing cardiac conditions. In some embodiments, the therapeutic agent includes, but is not limited to, angiotensin-converting enzyme (ACE) inhibitors (e.g., enalapril, lisinopril, and captopril), angiotensin II (A-II) receptor blockers (e.g., losartan and valsartan), diuretics (e.g., bumetanide, furosemide, and spironolactone), digoxin, beta blockers, and nesiritide.

In some embodiments, the scaffold further comprises cells (e.g., of the same or mixed cell types). In some embodiments, the cells are stem cells (e.g., cardiac stems cells or progenitors thereof).

In certain embodiments, the present invention provides methods of treating a cardiac condition, comprising contacting the heart of a subject suffering from a cardiac disorder with such a scaffold.

Such methods are not limited to treating a particular cardiac disorder. In some embodiments, the disorder is selected from the group consisting of chronic heart failure (CHF), ischemia without heart failure, cardiomyopathy, dilated cardiomyopathy (DCM), cardiac arrest, congestive heart failure, stable angina, unstable angina, myocardial infarction, coronary artery disease, valvular heart disease, ischemic heart disease, reduced ejection fraction, reduced myocardial perfusion, maladaptive cardiac remodeling, left ventricle remodeling, reduced left ventricle function, left heart failure, right heart failure, backward heart failure, forward heart failure, systolic dysfunction, diastolic dysfunction, systemic vascular resistance, low-output heart failure, high-output heart failure, dyspnea on exertion, dyspnea at rest, orthopnea, tachypnea, paroxysmal nocturnal dyspnea, dizziness, confusion, cool extremities at rest, exercise intolerance, easy fatigueability, peripheral edema, nocturia, ascites, hepatomegaly, pulmonary edema, cyanosis, laterally displaced apex beat, gallop rhythm, heart murmurs, parasternal heave, pleural effusion, congenital heart defects, and arrhythmia.

In some embodiments, the treating comprises one or more of improving right ventricular function, improving left ventricular function, improving right atrium function, improving left atrium function, fall in end diastolic pressure (EDP), improving myocardial perfusion, repopulating of the heart's anterior wall with cardiomyocytes, and reversing maladaptive left ventricle remodeling in CHF subjects.

In certain embodiments, the present invention provides methods of administering a therapeutic agent to a patient suffering from a cardiac disorder, comprising contacting the heart of a patient with such a scaffold, wherein the scaffold further comprises a therapeutic agent known to be useful in treating the cardiac condition.

In certain embodiments, the present invention provides methods of delivering stem cells or progenitors thereof to heart tissue, comprising contacting the heart tissue with such a scaffold, wherein the scaffold further comprises cardiac stem cells or progenitors thereof.

In certain embodiments, the present invention provides methods of delivering a medical device to heart tissue.

Additional embodiments are described herein.

### BRIEF DESCRIPTION OF THE DRAWINGS

FIG. 1 shows A): Image of dog bone mesh samples before tensile testing: polycarbonate co-polymer (top) polyglactin 910 (bottom) and B): Image of dog bone mesh samples after tensile testing: polyglactin 910 (left) and polycarbonate co-polymer (right).

FIG. 2 shows a graph of raw force (N) vs. displacement (mm) data from tensile testing (A), the slope of the linear portion of this curve (B) equates to material stiffness.

FIG. 3 shows a table of stiffness of different materials over time.

FIG. 4 shows Graphs of average material stiffness data over degradation showing a general decrease in stiffness. A) polycarbonate. B) polyglactin 910.

FIG. 5 shows a graph of average material maximum tensile strength data over degradation showing a general decrease in strength. A) polycarbonate. B) polyglactin 910.

FIG. 6 shows PV loop data showing that a restrictive material (Biomaterial A) (black squares) shifts the PV loop to the left compared to Sham (Green squares).

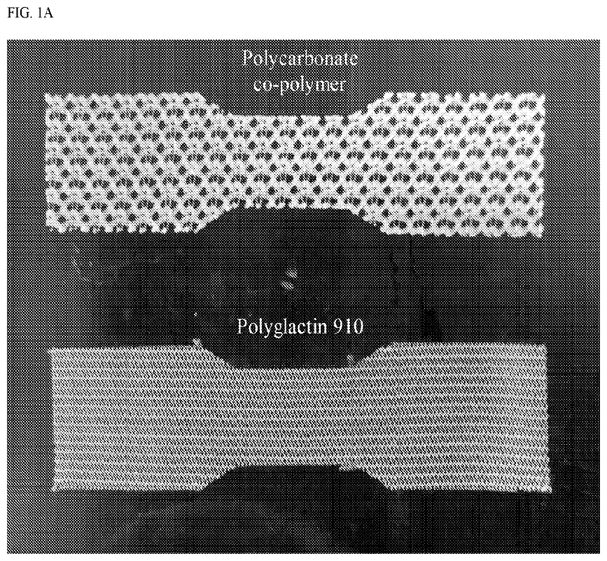

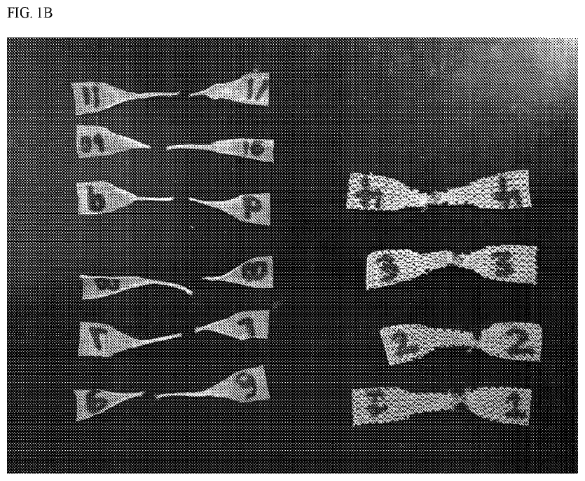

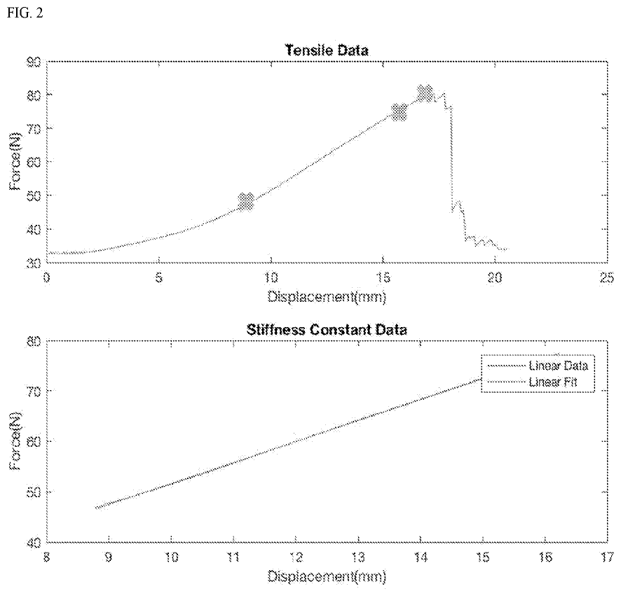

### DETAILED DESCRIPTION

Provided herein are scaffolds (e.g., synthetic meshes) having optimized material properties (e.g., initial stiffness, tensile strength) and related uses thereof (e.g., use in cardiac medical procedures).

Chronic heart failure (CHF) is one of the leading causes of death in the United States. One proposed approach to treating CHF is through the use of engineered tissues containing synthetic mesh material on which cells are seeded. For this work and other applications, it is useful to characterize the appropriate mechanical properties of these meshes, to support regeneration without impeding native tissue function. In cardiac applications, for example, a prolonged degradation period and high mechanical strength could restrict ventricular filling. Experiments described herein utilized three different degradation methods on existing, commercially available materials to tailor their mechanical properties post-manufacture. Tensile testing provides the ability to monitor and quantify mechanical strength and stiffness of such materials over the course of degradation. The present disclosure thus provides compositions and method for elucidating the material characteristics of meshes through tensile testing, in order to manipulate the mechanical properties of existing materials and confirm safety during implantation.

Accordingly, in certain embodiments, the present invention provides scaffolds configured for implanting in a human or animal body, wherein the scaffold has an initial stiffness value and initial mechanical tensile strength value optimized for cardiac application.

Such scaffolds are not limited to particular initial stiffness values and/or initial mechanical strength values. In some embodiments, the initial stiffness values and/or initial mechanical strength values enable the resulting scaffold to engage with heart tissue and not adversely affect native cardiac function. In some embodiments, the initial stiffness values and/or initial mechanical strength values prevent a prolonged degradation period. In some embodiments, the initial stiffness values and/or initial mechanical strength values prevent restriction of ventricular filling.

In some embodiments, the scaffold has an initial stiffness of at or lower than 40 N/mm (e.g., lower than 30.0, 10.0, 6.0, 4.0, 3.0, or 2.0).

In some embodiments, the scaffold has an initial mechanical tensile at or lower than approximately 85N.

In some embodiments, the scaffolds described herein does not shift the pressure-volume loop in vivo. Real-time left ventricular (LV) pressure-volume loops provide a framework for understanding cardiac mechanics in experimental animals and humans. Such loops can be generated by real-time measurement of pressure and volume within the left ventricle. Several physiologically relevant hemodynamic parameters such as stroke volume, cardiac output, ejection fraction, myocardial contractility, etc. can be determined from these loops. To generate a PV loop for the left ventricle, the LV pressure is plotted against LV volume at multiple time points during a single cardiac cycle.

In some embodiments, chamber stiffness is described as shifts in the diastolic pressure-volume (P/V) relationships compared to normal. When the P/V relationship is shifted toward the pressure axis, the left ventricle is stiffer, i.e., classically less compliant. The reverse is also true, when the P/V relationship is sifted away from the pressure axis, the left ventricle is classically more compliant. The clinical consequences are determined by the P/V relationship and the size of the left ventricle, meaning that just because the left ventricle is more compliant may not be good if the chamber is dilated. In some embodiments, the best clinical scenario is for the compliance of the left ventricle to be shifted back to normal.

In some embodiments, the stiffness and tensile strength of a material are assayed (e.g., using the methods described in Example 1) and the material is degraded in vitro to the desired stiffness and strength before use. For example, in some embodiments, degradation comprises one or more of hydrolytic degradation in a buffer solution at acidic pH (e.g., phosphate 1× buffer solution with a pH of 6.7 at 37° C.), chemical degradation (e.g., with ethylene oxide), and photolytic degradation (e.g., using UV light). In some embodiments, stiffness and tensile strength are monitored during degradation in order to obtain the optimal mechanical properties for the specific application.

The scaffolds described herein are scaffolded of any number of suitable materials. In some embodiments, the scaffold comprises synthetic material. In some embodiments, the scaffold comprises biological material. In some embodiments, the scaffold is a hybrid of synthetic and biological materials.

Examples of suitable scaffold material include, but are not limited to, one or more of collagen, fibronectin, polyglycolides, polylactides, polypropylene, polyester, silicone, expanded polytetrafluorothylene, Dexon, Vicryl, polycaprolactone, polydioxanone, catgut, silk, nylon, and trimethylene carbonate.

For certain application, the scaffold is composed of a polylactide material or a polyglactin 910 material. In some embodiments, the scaffold is derived from human, bovine or porcine tissue.

In some embodiments, the scaffold is bioabsorbable. In some embodiments, the scaffold is non-bioabsorbable.

For certain applications, the scaffold further comprises a therapeutic agent such as a drug or biologic. In some embodiments, the therapeutic agent is known to be useful in treating, ameliorating and/or preventing cardiac conditions. Examples include, but are not limited to, angiotensin-converting enzyme (ACE) inhibitors (e.g., enalapril, lisinopril, and captopril), angiotensin II (A-II) receptor blockers (e.g., losartan and valsartan), diuretics (e.g., bumetanide, furosemide, and spironolactone), digoxin, beta blockers, and nesiritide.

In some embodiments, the scaffold further comprises cells (e.g., of the same or mixed cell types). In some embodiments, the cells are stem cells (e.g., cardiac stems cells or progenitors thereof).

In certain embodiments, the present invention provides methods of treating a cardiac condition, comprising contacting the heart of a subject suffering from a cardiac disorder with such a scaffold.

Such methods are not limited to treating a particular cardiac disorder. In some embodiments, the disorder is selected from the group consisting of chronic heart failure (CHF), ischemia without heart failure, cardiomyopathy, dilated cardiomyopathy (DCM), cardiac arrest, congestive heart failure, stable angina, unstable angina, myocardial infarction, coronary artery disease, valvular heart disease, ischemic heart disease, reduced ejection fraction, reduced myocardial perfusion, maladaptive cardiac remodeling, left ventricle remodeling, reduced left ventricle function, left heart failure, right heart failure, backward heart failure, forward heart failure, systolic dysfunction, diastolic dysfunction, systemic vascular resistance, low-output heart failure, high-output heart failure, dyspnea on exertion, dyspnea at rest, orthopnea, tachypnea, paroxysmal nocturnal dyspnea, dizziness, confusion, cool extremities at rest, exercise intolerance, easy fatigueability, peripheral edema, nocturia, ascites, hepatomegaly, pulmonary edema, cyanosis, laterally displaced apex beat, gallop rhythm, heart murmurs, parasternal heave, pleural effusion, congenital heart defects, and arrhythmia.

As used herein, “CHF” is a chronic (as opposed to rapid onset) impairment of the heart's ability to supply adequate blood to meet the body's needs. CHF may be caused by, but is distinct from, cardiac arrest, myocardial infarction, and cardiomyopathy. In one alternative embodiment, the subject suffers from congestive heart failure. In various further alternative embodiments that can be combined with any other embodiments herein, the subject's heart failure comprises left heart failure, right heart failure, backward heart failure (increased venous back pressure), forward heart failure (failure to supply adequate arterial perfusion), systolic dysfunction, diastolic dysfunction, systemic vascular resistance, low-output heart failure, high-output heart failure. In various further alternative embodiments that can be combined with any other embodiments herein, the subject's CHF may be any of Classes I-IV as per the New York Heart Association Functional Classification; more preferably Class III or IV.

Class I: no limitation is experienced in any activities; there are no symptoms from ordinary activities.
Class II: slight, mild limitation of activity; the patient is comfortable at rest or with mild exertion.
Class III: marked limitation of any activity; the patient is comfortable only at rest.
Class IV: any physical activity brings on discomfort and symptoms occur at rest.

In a further alternative embodiment that can be combined with any other embodiments herein, the subject has been diagnosed with CHF according to the New York Heart Association Functional Classification. In a further alternative embodiment that can be combined with any other embodiments herein, the subject is further characterized by one or more of the following: hypertension, obesity, cigarette smoking, diabetes, valvular heart disease, and ischemic heart disease.

As used herein, “treat” or “treating” means accomplishing one or more of the following: (a) reducing the severity of the disorder (ex: treatment of Class IV subject to improve status to Class III for CHF subjects); (b) limiting or preventing development of symptoms characteristic of the disorder; (c) inhibiting worsening of symptoms characteristic of the disorder; (d) limiting or preventing recurrence of symptoms in patients that were previously symptomatic for the disorder; and (e) increasing life span (e.g., improving mortality). Signs characteristic of CHF include, but are not limited to reduced ejection fraction, reduced myocardial perfusion, maladaptive cardiac remodeling (such as left ventricle remodeling), reduced left ventricle function, dyspnea on exertion, dyspnea at rest, orthopnea, tachypnea, paroxysmal nocturnal dyspnea, dizziness, confusion, cool extremities at rest, exercise intolerance, easy fatigueability, peripheral edema, nocturia, ascites, hepatomegaly, pulmonary edema, cyanosis, laterally displaced apex beat, gallop rhythm, heart murmurs, parasternal heave, and pleural effusion.

In some embodiments, the treating comprises one or more of improving right ventricular function, improving left ventricular function, fall in end diastolic pressure (EDP), improving myocardial perfusion, repopulating of the heart's anterior wall with cardiomyocytes, and reversing maladaptive left ventricle remodeling in CHF subjects.

In certain embodiments, the present invention provides methods of administering a therapeutic agent to a patient suffering from a cardiac disorder, comprising contacting the heart of a patient with such a scaffold, wherein the scaffold further comprises a therapeutic agent known to be useful in treating the cardiac condition.

In certain embodiments, the present invention provides methods of delivering stem cells or progenitors thereof to heart tissue, comprising contacting the heart tissue with such a scaffold, wherein the scaffold further comprises stem cells (e.g., cardiac stem cells) or progenitors thereof.

In certain embodiments, the present invention provides methods of delivering a medical device to heart tissue.

The scaffold can be contacted with the heart in any suitable way to promote attachment. The scaffold may be attached to various locations on the heart, including the epicardium, myocardium and endocardium, most preferably the epicardium. Means for attachment include, but are not limited to, direct adherence between the scaffold and the heart tissue, biological glue, suture, synthetic glue, laser dyes, or hydrogel. A number of commercially available hemostatic agents and sealants include SURGICAL® (oxidized cellulose), ACTIFOAM® (collagen), FIBRX® (light-activated fibrin sealant), BOHEAL® (fibrin sealant), FIBROCAPS® (dry powder fibrin sealant), polysaccharide polymers p-GlcNAc (SYVEC® patch; Marine Polymer Technologies), Polymer 27CK (Protein Polymer Tech.). Medical devices and apparatus for preparing autologous fibrin sealants from 120 ml of a patient's blood in the operating room in one and one-half hour are also known (e.g. Vivostat System).

In an alternative embodiment of the invention utilizing direct adherence, the scaffold is placed directly onto the heart and the product attaches via natural cellular attachment. In a further alternative embodiment, the scaffold is attached to the heart using surgical glue, preferably biological glue such as a fibrin glue. The use of fibrin glue as a surgical adhesive is well known. Fibrin glue compositions are known (e.g., see U.S. Pat. Nos. 4,414,971; 4,627,879 and 5,290,552) and the derived fibrin may be autologous (e.g., see U.S. Pat. No. 5,643,192). The glue compositions may also include additional components, such as liposomes containing one or more agent or drug (e.g., see U.S. Pat. Nos. 4,359,049 and 5,605,541) and include via injection (e.g., see U.S. Pat. No. 4,874,368) or by spraying (e.g., see U.S. Pat. Nos. 5,368,563 and 5,759,171). Kits are also available for applying fibrin glue compositions (e.g., see U.S. Pat. No. 5,318,524).

In another embodiment, a laser dye is applied to the heart, the scaffold, or both, and activated using a laser of the appropriate wavelength to adhere to the tissues. In alternative embodiments, the laser dye has an activation frequency in a range that does not alter tissue function or integrity. For instance, 800 nm light passes through tissues and red blood cells. Using indocyan green (ICG) as the laser dye, laser wavelengths that pass through tissue may be used. A solution of 5 mg/ml of ICG is painted onto the surface of the three-dimensional stromal tissue (or target site) and the ICG binds to the collagen of the tissue. A 5 ms pulse from a laser emitting light with a peak intensity near 800 nm is used to activate the laser dye, resulting in the denaturation of collagen which fuses elastin of the adjacent tissue to the modified surface.

In another embodiment, the scaffold is attached to the heart using a hydrogel. A number of natural and synthetic polymeric materials are sufficient for forming suitable hydrogel compositions. For example, polysaccharides, e.g., alginate, may be crosslinked with divalent cations, polyphosphazenes and polyacrylates are crosslinked ionically or by ultraviolet polymerization (U.S. Pat. No. 5,709,854). Alternatively, a synthetic surgical glue such as 2-octyl cyanoacrylate (“DERMABOND™”, Ethicon, Inc., Somerville, N.J.) may be used to attach the three-dimensional stromal tissue.

In an alternative embodiment of the present invention, the scaffold is secured to the heart using one or more sutures, including, but not limited to, 5-0, 6-0 and 7-0 proline sutures (Ethicon Cat. Nos. 8713H, 8714H and 8701H), poliglecaprone, polydioxanone, polyglactin or other suitable non-biodegradable or biodegradable suture material. When suturing, double armed needles are typically, although not necessarily, used.

The methods and compositions described herein can be used in combination with conventional treatments, such as the administration of various pharmaceutical agents and surgical procedures. Medications suitable for use in the methods described herein include angiotensin-converting enzyme (ACE) inhibitors (e.g., enalapril, lisinopril, and captopril), angiotensin II (A-II) receptor blockers (e.g., losartan and valsartan), diuretics (e.g., bumetanide, furosemide, and spironolactone), digoxin, beta blockers, and nesiritide.

A number of methods can be used to measure changes in the functioning of the heart in subjects before and after attachment of the scaffold. For example, an echocardiogram can be used to determine the capacity at which the heart is pumping. The percentage of blood pumped out of the left ventricle with each heartbeat is referred to as the ejection fraction. In a healthy heart, the ejection fraction is about 60 percent. In an individual with chronic heart failure caused by the inability of the left ventricle to contract vigorously, i.e., systolic heart failure, the ejection fraction is usually less than 40 percent. Depending on the severity and cause of the heart failure, ejection fractions typically range from less than 40 percent to 15 percent or less. An echocardiogram can also be used to distinguish between systolic heart failure and diastolic heart failure, in which the pumping function is normal but the heart is stiff.

In some embodiments, echocardiograms are used to compare the ejection fractions before and following treatment with the scaffold. In certain embodiments, treatment with the scaffold results in improvements in the ejection fraction between 3 to 5 percent. In other embodiments, treatment with the scaffold results in improvements in the ejection fraction between 5 to 10 percent. In still other embodiments, treatment with the scaffold results in improvements in the ejection fraction greater than 10 percent.

Nuclear scans, such as radionuclide ventriculography (RNV) or multiple gated acquisition (MUGA) scanning can be used to determine how much blood the heart pumps with each beat. These tests are done using a small amount of dye injected in the veins of an individual A special camera is used to detect the radioactive material as it flows through the heart. Other tests include X-rays, MRI, and blood tests. Chest X-rays can be used to determine the size of the heart and if fluid has accumulated in the lungs. Blood tests can be used to check for a specific indicator of congestive heart failure, brain natriuretic peptide (BNP). BNP is secreted by the heart in high levels when it is overworked. Thus, changes in the level of BNP in the blood can be used to monitor the efficacy of the treatment regime.

In a further aspect, the present invention provides kits for treating a heart disorder (e.g., CHF), comprising a suitable scaffold as disclosed above and a means for attaching the scaffold to the heart or organ. The means for attachment may include any such attachment device as described above, for example, a composition of surgical glue, hydrogel, or preloaded prolene needles for microsuturing.

### EXPERIMENTAL

The following examples are provided to demonstrate and further illustrate certain embodiments of the present disclosure and are not to be construed as limiting the scope thereof.

### Example 1

Based on previous in vivo left ventricular (LV) pressure-volume data acquired from terminal hemodynamic studies conducted on Sprague-Dawley rats with left coronary artery ligation to create chronic heart failure, the implanted cultured polycarbonate material was determined to no longer be cardio-restrictive at 21 days post implantation.

### Materials and Methods

Mimicking in vivo degradation conditions, two mesh materials: (1) a two layer copolymer of polyglycolide and polylactide fibers and (2) polyglactin 910, were hydrolytically degraded in a 6.7 pH phosphate buffer at 37 C.

Hydrolytic Degradation: the samples were degraded in vitro in a phosphate 1× buffer solution with a pH of 6.7 at 37° C. to mimic a pH and temperature suitable for cell culture. Samples were continuously degraded and tensile tested (n=5) at fourteen-day intervals for the first 28 days, and seven-day intervals for the following 28 days.

Chemical Degradation: Ethylene oxide sterilization included an initial purge cycle (to remove excess air from the sterilization chamber), a twelve-hour sterilization cycle, and a two hour aeration/de-gassing cycle post sterilization. Samples were tested (n=5) twelve hours after 3, 5, 7, and 10 complete sterilizations had been completed.

Photolytic Degradation: the samples were degraded under 254 nm UV light at an intensity of approximately 15 mW/cm2. The degradation set-up was hemispheric with the interior covered in reflective foil to maintain uniformity in intensity. Samples were exposed to the UV light in twelve-hour intervals and tensile tested (n=5) after each degradation interval.

Tensile testing was performed using a MTS Criterion® Series 40 Electromechanical Universal Test Systems Model 42. The test rate was set at 0.5 mm/min and the sample width was set at 10 mm.

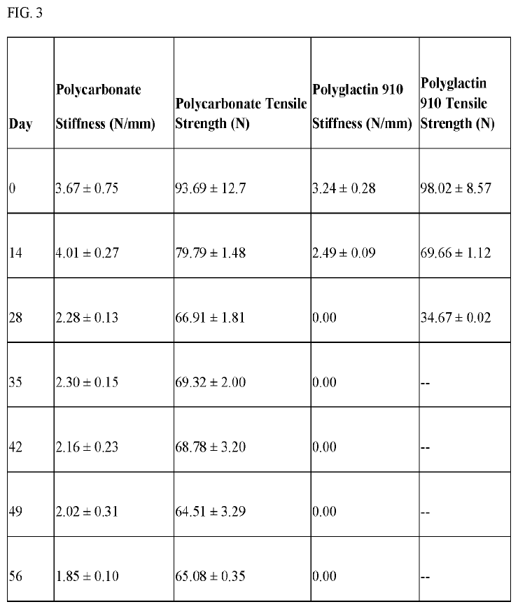

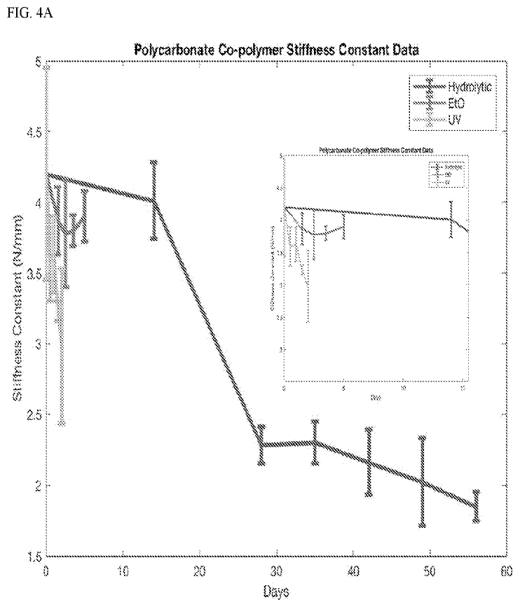

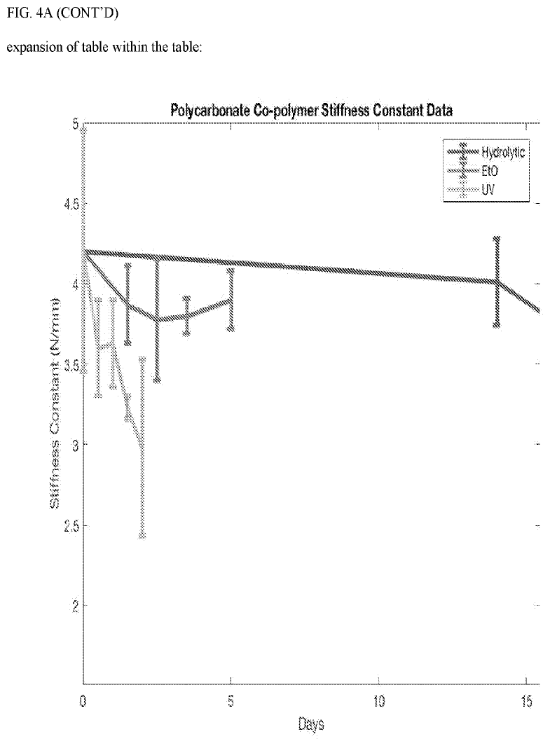

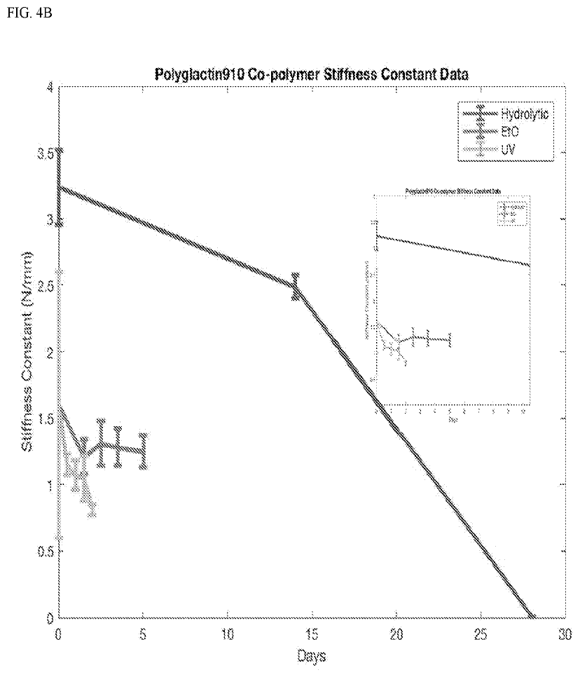

Stiffness constants were measured from tensile data (FIG. 2) and compared to in vivo pressure volume measurements to identify ideal tensile strength and stiffness (k) for safety.

### Results

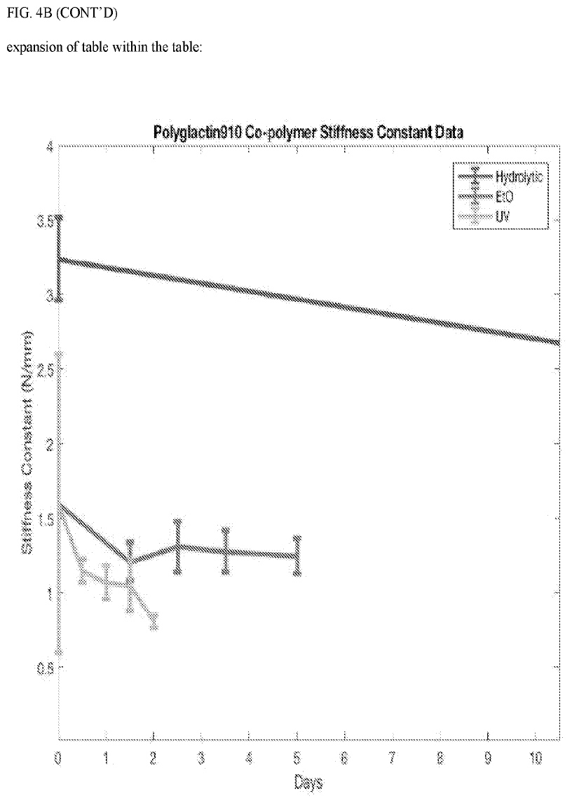

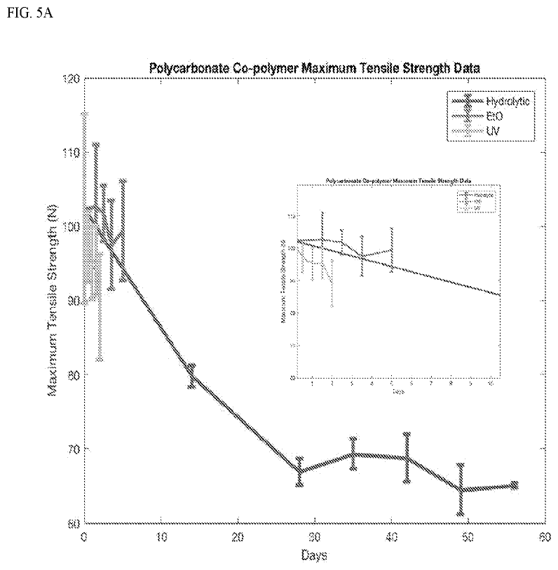

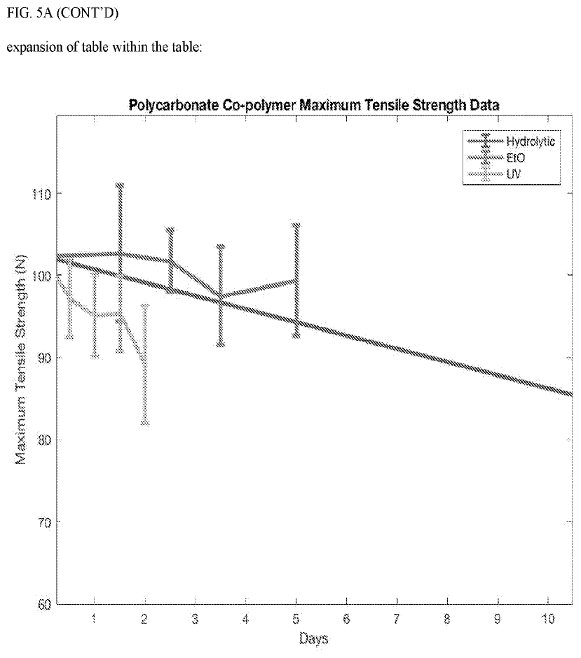

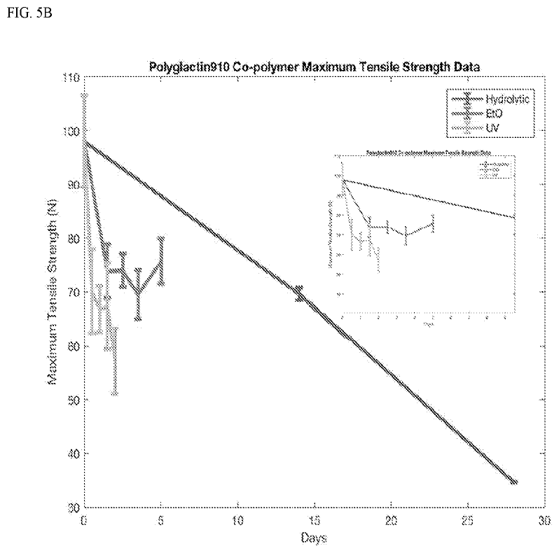

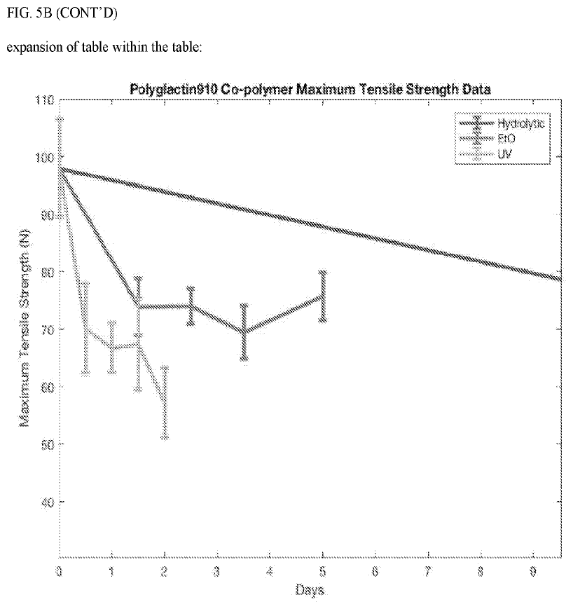

Initial stiffness and maximum tensile strength of the polycarbonate material (n=5) was (3.67±0.75) newtons/mm (N/mm) and (93.7±12.7) N, respectively. In vitro degradation of the same material corresponded to a material stiffness and a maximum tensile strength of 2.02±0.31 N/mm and 64.50±3.29 N (hydrolytic), 3.90±0.18 N/mm and 99.38±6.70 N (chemical), and 2.98±0.55 N/mm and 89.13±7.14 N (photolytic) (FIGS. 4,5).

Initial stiffness and maximum tensile strength of polyglactin 910 material (n=5) was (3.24±0.28) N/mm and (98.0±8.57) N, respectively. In vitro degradation of the same material corresponded to a material stiffness and a maximum tensile strength of 2.02±0.31 N/mm and 64.5±3.29 N (hydrolytic), 1.25±0.12 N/mm and 75.77±4.20 N (chemical), and 0.81±0.04 N/mm and 57.20±6.07 N (photolytic) (FIGS. 4,5).

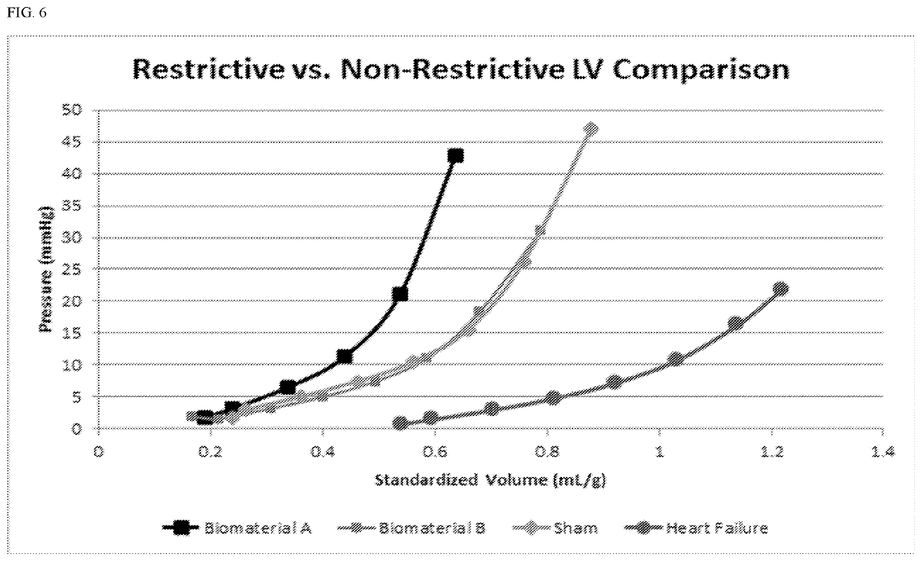

FIG. 6 shows PV loop data showing that a restrictive material (Biomaterial A; n=3) (black squares) shifts the PV loop to the left compared to Sham (Green squares; n=9). This demonstrates that means that the Biomaterial A compromises the filling of the left ventricle; the Biomaterial A is restrictive. The clinical translation is that the restrictive ventricle ends up smaller and does not fill normally.

This example demonstrates the ability to manipulate the mechanical properties of synthetic meshes in-vitro to tailor the material and prevent cardio-restriction. Prior in vivo testing of the polycarbonate co-polymer material indicated that the material ceased to be cardio-restrictive (Biomaterial B). In vitro hydrolytic degradation data indicates that this time point corresponds to an approximate material stiffness and maximum tensile strength of 3.25 N/mm and 70 N, respectively. The results of this study support that photolytic degradation however, may deliver the same magnitude of degradation as hydrolytic degradation in a shorter time span.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A scaffold configured for implanting in a human or animal body, wherein the scaffold has an initial stiffness value and initial mechanical tensile strength value optimized for cardiac application.

2. The scaffold of claim 1, wherein the scaffold does not shift the pressure-volume loop toward the pressure axis from normal

3. The scaffold of claim 1, wherein the scaffold has an initial stiffness of at or lower than 40.0 N/mm.

4-8. (canceled)

9. The scaffold of claim 1, wherein the scaffold has an initial stiffness of at or lower than 2.0 N/mm.

10. The scaffold of claim 1, wherein the scaffold comprises synthetic material.

11. The scaffold of claim 1, wherein the scaffold comprises biological material.

12. The scaffold of claim 10, wherein the scaffold is composed of one or more of collagen, fibronectin, polyglycolides, polylactides, polypropylene, polyester, silicone, polycarbonate, expanded polytetrafluorothylene, Dexon, Vicryl, polycaprolactone, polydioxanone, catgut, silk, nylon, and trimethylene carbonate in a three-dimensional matrix.

13-17. (canceled)

17. The scaffold of claim 11, wherein the scaffold is derived from human, bovine or porcine tissue.

18. The scaffold of claim 1, wherein the scaffold is absorbable or non-absorbable.

19-20. (canceled)

21. The scaffold of claim 1, wherein the scaffold further comprises a therapeutic agent.

22. The scaffold of claim 21, wherein the therapeutic agent is known to be useful in treating, ameliorating and/or preventing cardiac conditions.

23. The scaffold of claim 1, further comprising a plurality of cells.

24. The scaffold of claim 23, wherein said cells are the same or different types.

25. The scaffold of claim 23, wherein said cells are stem cells or progenitors thereof.

26. The scaffold of claim 25, wherein said cells cardiac stems cells or progenitors thereof.

27. A method of treating a cardiac condition, comprising contacting the heart of a subject suffering from a cardiac disorder with a scaffold of claim 1.

28. The method of claim 27, wherein the disorder is selected from the group consisting of chronic heart failure (CHF), ischemia without heart failure, cardiomyopathy, dilated cardiomyopathy (DCM), cardiac arrest, congestive heart failure, stable angina, unstable angina, myocardial infarction, coronary artery disease, valvular heart disease, ischemic heart disease, reduced ejection fraction, reduced myocardial perfusion, maladaptive cardiac remodeling, left ventricle remodeling, reduced left ventricle function, left heart failure, right heart failure, backward heart failure, forward heart failure, systolic dysfunction, diastolic dysfunction, systemic vascular resistance, low-output heart failure, high-output heart failure, dyspnea on exertion, dyspnea at rest, orthopnea, tachypnea, paroxysmal nocturnal dyspnea, dizziness, confusion, cool extremities at rest, exercise intolerance, easy fatigueability, peripheral edema, nocturia, ascites, hepatomegaly, pulmonary edema, cyanosis, laterally displaced apex beat, gallop rhythm, heart murmurs, parasternal heave, pleural effusion, congenital heart defects, and arrhythmia.

29. The method of claim 27, wherein the scaffold adheres to the heart.

30. The method of claim 27, wherein the treating comprises one or more of improving electrical signaling, improving right ventricle function, improves left ventricular function, improving right atrium function, improving left atrium function, treating congenital defects, fall in end diastolic pressure (EDP), improving myocardial perfusion, repopulating of the heart's anterior wall with cardiomyocytes, and reversing maladaptive left ventricle remodeling.

31. A method of administering a therapeutic agent to a patient suffering from a cardiac disorder, comprising contacting the heart of a patient with a scaffold of claim 1, wherein the scaffold further comprises a therapeutic agent known to be useful in treating the cardiac condition.

32-35. (canceled)
